(19) United States
(12) Patent Application Publication
(10) Pub. No.: US 2023/0114833 A1
(43) Pub. Date: Apr. 13, 2023

(54) TECHNIQUES FOR MEASURING HEART RATE DURING EXERCISE

(71) Applicant: Oura Health Oy, Oulu (FI)

(72) Inventors: Xi Zhang, Daly City, CA (US); Ronny Li, San Francisco, CA (US); Antti Aleksi Rantanen, Oulu (FI); Tero Juhani Vallius, Kontio (FI); Jussi Petteri Järvelä, Kempele (FI); Gerald Pho, Somerville, MA (US)

(21) Appl. No.: 17/954,564

(22) Filed: Sep. 28, 2022

Related U.S. Application Data
(60) Provisional application No. 63/254,849, filed on Oct. 12, 2021.

(51) Int. Cl. A61B 5/00; A61B 5/024; A61B 5/11; A61B 5/026

(52) U.S. Cl. CPC A61B 5/721; A61B 5/02427; A61B 5/11; A61B 5/7264; A61B 5/742; A61B 5/7475; A61B 5/6802; A61B 5/0261; A61B 5/02438; A61B 2562/0219

(57) ABSTRACT

Methods, systems, and devices for heart rate detection are described. A method for measuring heart rate for a user may include receiving physiological data associated with the user, wherein the physiological data may include photoplethysmogram (PPG) data and motion data collected throughout a first time interval via a wearable device associated with the user. The method may include determining a set of candidate heart rate measurements within the first time interval based at least in part on the PPG data, selecting a first heart rate measurement from the set of candidate heart rate measurements based on the received motion data, and determining a first heart rate for the user within the first time interval based on the selected first heart rate measurement.

### CROSS REFERENCE

The present application for patent claims the benefit of U.S. Provisional Patent Application No. 63/254,849 by ZHANG et al., entitled “TECHNIQUES FOR MEASURING HEART RATE DURING EXERCISE,” filed Oct. 12, 2021, assigned to the assignee hereof, and expressly incorporated by reference herein.

### FIELD OF TECHNOLOGY

The following relates to wearable devices and data processing, including techniques for measuring heart rate during exercise.

### BACKGROUND

Some wearable devices may be configured to collect data from users associated with heart rate of the user, such as motion data, temperature data, photoplethysmogram (PPG) data, etc. In some cases, some wearable devices may be unable to accurately determine heart rate data of a user, such as while the user is exercising or otherwise moving.

### BRIEF DESCRIPTION OF THE DRAWINGS

FIG. 1 illustrates an example of a system that supports techniques for measuring heart rate in accordance with aspects of the present disclosure.

FIG. 2 illustrates an example of a system that supports techniques for measuring heart rate in accordance with aspects of the present disclosure.

FIG. 3 illustrates an example of a heart rate determination procedure that supports techniques for measuring heart rate in accordance with aspects of the present disclosure.

FIG. 4 illustrates an example of a heart rate determination procedure that supports techniques for measuring heart rate in accordance with aspects of the present disclosure.

FIG. 5 illustrates an example of a graphical user interface (GUI) that supports techniques for measuring heart rate in accordance with aspects of the present disclosure.

FIG. 6 shows a block diagram of an apparatus that supports techniques for measuring heart rate in accordance with aspects of the present disclosure.

FIG. 7 shows a block diagram of a wearable application that supports techniques for measuring heart rate in accordance with aspects of the present disclosure.

FIG. 8 shows a diagram of a system including a device that supports techniques for measuring heart rate in accordance with aspects of the present disclosure.

FIGS. 9 through 11 show flowcharts illustrating methods that support techniques for measuring heart rate in accordance with aspects of the present disclosure.

### DETAILED DESCRIPTION

A user may use a device (e.g., a wearable device) to determine physiological measurements of the user, such as heart rate. Some wearable devices may use photoplethysmogram (PPG) data to determine a heart rate for a user over time. For example, a wearable device may measure heart rate of a user by detecting changes in blood pulse volume through PPG sensors in the wearable device (e.g., infrared PPG sensors, infrared light emitting diodes (LEDs)). Each time that a user's heart beats, blood is pumped out to the arteries located in the user's hands and fingers. The PPG sensors are able to detect these changes in blood flow and volume using light reflection and absorption. Each pulse causes the arteries in a user's finger to alternate between swelling and contracting. By shining a light on the skin of the user, particularly on the skin of a finger, changes in light absorbed by the blood and reflected back from the wavering volume of red blood cells in the arteries is measured. From here, PPG can represent these blood flow changes through a visual waveform that represents the activity of the user's heart (e.g., heart rate).

Heart rate is a sensitive metric that is subject to change based on activities being performed by a user (e.g., drinking a glass of water, standing up, watching television, exercise). Certain activities may result in a spike or dip in heart rate, and such variations may be referred to as “noise” in the data. Additionally, some activities may produce a PPG signal that may be misinterpreted as a heart rate signal. For example, exercises such as running, jumping, etc. may cause motion artifacts (e.g., a false PPG signal). The optical nature of PPG sensors may render the PPG measurements susceptible to motion artifacts from variable and discontinuous contact between the device and skin. Motion artifacts are typically caused by the change of blood flow velocity induced by the motion of the exercise or the relative movement between PPG sensors and the skin of the user. For example, some motions (e.g., running) may result in a periodic pressure between the wearable device (e.g., the sensors of the wearable device) and the part of the user that the wearable device is positioned, and as such may apply periodic pressure to blood vessels. The periodic pressure caused by the periodic motion may cause the blood vessels to periodically constrict and dilate. Accordingly, the wearable device may detect such constriction and dilation of the vessels as an artificial PPG signal because the constriction and dilation were due to the motion of the user, and not due to the heart rate of the user. Such artificial PPG signals that are attributable to motion may be referred to as “motion artifacts.”

As such, due to these difficulties with measuring heart rate during exercise or other motion, some conventional wearable devices may not track a user's heart rate throughout the day and/or during periods of motion accurately, or at all. In such cases, the wearable device may provide only a limited depiction of the user's heart rate, and therefore a limited depiction of the user's overall health. Additionally, heart rate may be a valuable tool to a user during an exercise because heart rate may provide an indication of effort and intensity during the exercise. For example, a user may utilize heart rate during an exercise to determine whether to increase or decrease intensity of a workout. Accordingly, it may be beneficial to implement techniques to accurately determine heart rate of a user during periods of activity, such as during an exercise. The wearable devices described herein may be configured with a procedure for detecting heart data and distinguishing the actual heart rate data of the user from motion artifacts impacting the data.

Accordingly, aspects of the present disclosure are directed to techniques for measuring a heart rate of a user in such a manner that is less susceptible to motion and other noise. A procedure for determining heart rate may include receiving physiological data associated with the user, wherein the physiological data may include PPG data and motion data (e.g., acceleration data) collected throughout a first time interval via a wearable device associated with the user. In some cases, the motion data may refer to acceleration data and may be used to determine periods of motion of the user. The method may include determining a set of candidate heart rate measurements within the first time interval based at least in part on the PPG data. The set of candidate heart rate measurements may include artificial heart rate measurements (e.g., heart rate measurements that may be attributable to periodic motion or other activity). Accordingly, the method may include selecting a first heart rate measurement from the set of candidate heart rate measurements based on the received motion data. For example, the set of candidate heart rate measurements may be compared to the motion data to determine the candidate heart rate measurements that are correlated with motion. Thus, the heart rate data that is not due to motion may be selected. The method may be used to determine a first heart rate for the user within the first time interval based on the selected first heart rate measurement.

In some implementations, machine learning models or algorithms (e.g., heuristic-based models, deep learning models, regression-based models) may be used to determine heart rate measurements for the user. For example, a wearable device may acquire PPG data and motion data associated with a user. The acquired PPG and motion data may be input into a machine learning model that is configured to output a determined or estimated heart rate for the user. For instance, the machine learning model may be configured to differentiate between candidate heart rate measurements that are attributable to motion artifacts from candidate heart rate measurements that are indicative of the user's actual heart rate. In such cases, the machine learning model may be configured to select (e.g., identify, estimate) a candidate heart rate measurement, and determine a heart rate for the user based on the selected/estimated candidate heart rate measurements. By way of another example, the machine learning model may be configured to identify time-domain and/or frequency-domain features within the received PPG data and motion data (which may be used to identify candidate heart rate measurements), and may be configured to determine or estimate a heart rate for the user based on the identified features.

The procedures described herein may not be limited to determining heart rate during exercise. In some cases, the procedures described herein may be utilized to detect heart rate at all times, or regardless of activity level such that the wearable device may utilize the described techniques during periods of rest, activity, exercise, sleep, etc. Accordingly, particular aspects of the subject matter described herein may be implemented to realize one or more advantages. The described techniques may support improvements in detecting and determining heart rate data of a user to provide the user with comprehensive heart rate data during periods of rest, activity, exercise, sleep, etc.

Aspects of the disclosure are initially described in the context of systems supporting physiological data collection from users via wearable devices. Additional aspects of the disclosure are additionally described in the context of example heart rate determination procedures and an example graphical user interface (GUI). Aspects of the disclosure are further illustrated by and described with reference to apparatus diagrams, system diagrams, and flowcharts that relate to techniques for measuring heart rate during exercise.

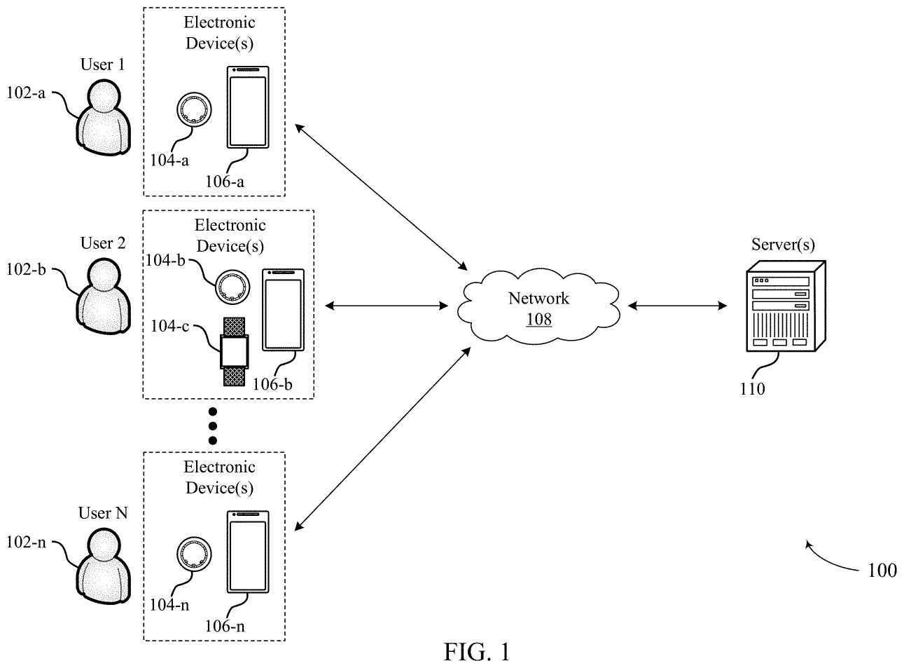

FIG. 1 illustrates an example of a system 100 that supports techniques for measuring heart rate in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, which may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) that emit light on the palm-side of a user's finger to collect physiological data based on arterial blood flow within the user's finger. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs which are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, which repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for determining heart rate data of a user based on physiological data (e.g., motion data) collected by a wearable device. The system may support techniques for determining heart rate data during periods of activity, motion, exercise, etc. In particular, the system 100 illustrated in FIG. 1 may support techniques for determining heart rate data of a user 102, and causing a user device 106 corresponding to the user 102 to display an indication of the heart rate data. For example, as shown in FIG. 1, User 1 (user 102-a) may be associated with a wearable device 104-a (e.g., ring 104-a) and a user device 106-a. In this example, the ring 104-a may collect data associated with the user 102-a, including motion, temperature, heart rate, HRV, and the like. In some aspects, the ring 104-a may be used to collect physiological data of the user that the ring 104-a may use to select actual heart rate data from motion artifacts. The ring 104-a may determine heart rate data based on the PPG monitoring.

Physiological data collection, PPG monitoring, and heart rate data determination may be performed by any of the components of the system 100, including the ring 104-a, the user device 106-a associated with User 1, the one or more servers 110, or any combination thereof. For example, in some implementations, collected PPG data and motion data may be inputted into a machine learning model that is configured to determine a heart rate for the user. Upon determination of heart rate data, the system 100 may selectively cause the GUI of the user device 106-a to display all or a subset of the heart rate data.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

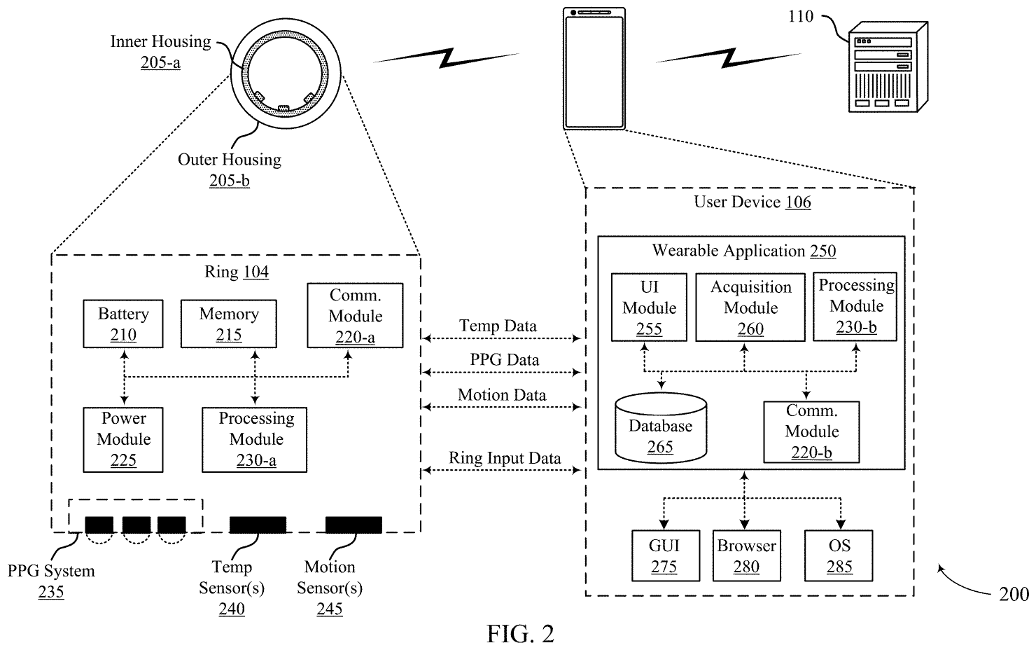

FIG. 2 illustrates an example of a system 200 that supports techniques for measuring heart rate in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, which may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, which may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user's finger. In some implementations, the PPG system 235 may be configured as a transmissive PPG system 235 in which the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, which may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for determining heart rate data of a user based on physiological data (e.g., motion data) collected by a wearable device. In some aspects, the ring 104, user device 106, and servers 110 of the system 200 may be configured to determine heart rate data of a user during periods of motion, activity, exercise, etc. In particular, the respective components of the system 200 may be used to determine heart rate data (e.g., exercise heart rate data) of a user based on physiological data of the user (e.g., motion). For example, the respective components of the system 200 may collect physiological data of the user that the ring 104-a (or other components of system 200) may use to select actual heart rate data and remove or ignore motion artifacts that may result in false heart rate measurements. As such, the physiological data may be obtained by leveraging sensors on the ring 104 of the system 200.

For example, as noted previously herein, the ring 104 of the system 200 may be worn by a user to collect data from the user, including temperature, heart rate, movement, and the like. The ring 104 of the system 200 may collect the physiological data from the user based on arterial blood flow. Physiological data collected by the ring 104 may be used to determine false or erroneous heart rate data that is due to motion (e.g., motion artifacts), rather than actual blood flow and accordingly, may be used to determine the actual heart rate data.

For instance, in some implementations, acquired PPG data and motion data may be inputted into a machine learning model (e.g., heuristic-based model, deep learning model, regression-based model) that is configured to determine a heart rate for the user. In such cases, the machine learning model may be configured to differentiate between candidate heart rate measurements that are attributable to motion artifacts from candidate heart rate measurements that are indicative of the user's actual heart rate. Additionally, or alternatively, the machine learning model may be configured to identify time-domain and/or frequency-domain features within the received PPG data and motion data (which may be used to identify candidate heart rate measurements), and may be configured to determine or estimate a heart rate for the user based on the identified features.

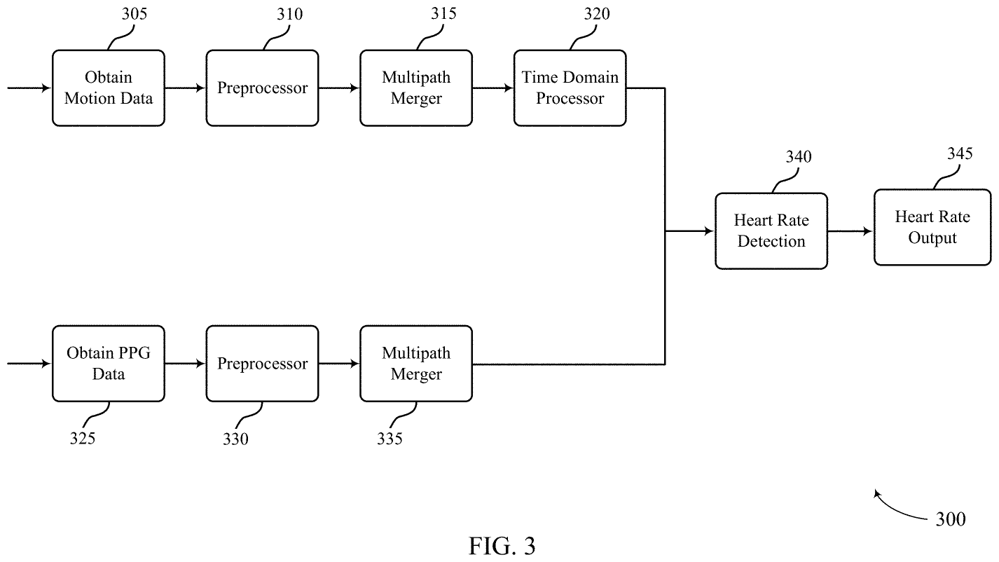

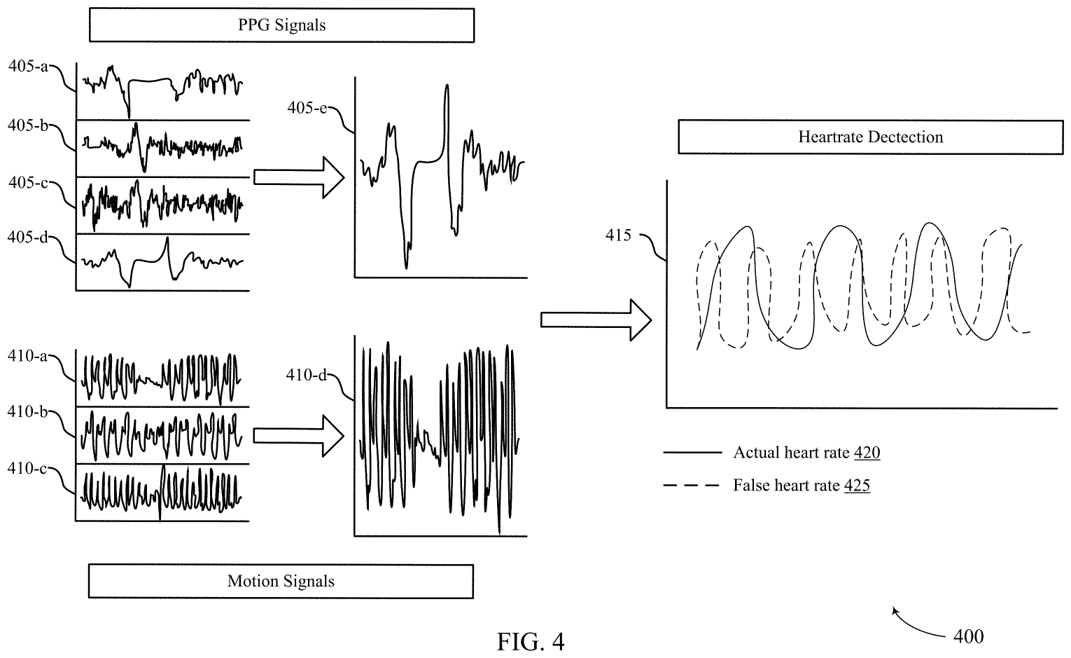

The procedure for determining heart rate data (e.g., exercise heart rate data) may be further shown and described with reference to FIGS. 3 and 4.

FIG. 3 illustrates an example of a heart rate determination procedure 300 that supports techniques for measuring heart rate in accordance with aspects of the present disclosure. The heart rate determination procedure 300 may implement, or be implemented by, aspects of the system 100, system 200, or a combination thereof. For example, in some implementations, the heart rate determination procedure 300 may result in heart rate data (e.g., active heart rate data, exercise heart rate data) that may be displayed to a user via the GUI 275 of the user device 106, as shown in FIG. 2.

As described herein, a system (e.g., system 200), or a portion of system 200, such as a wearable device (e.g., a ring 104), may identify heart rate data of a user from a set of heart rate data, where the heart rate data may include motion artifacts. In some cases, a wearable device may detect heart rate data of a user in accordance with the techniques described herein during periods of activity, motion, exercise, etc. In some cases, the techniques for determining heart rate data described herein may be utilized for heart rate detections other than active heart rate detections, or for all heart rate detections (e.g., despite whether the user is active or exercising). Accordingly, the heart rate determination procedure 300 may not be limited to determining heart rate of a user during exercise, motion, activity, etc. In some cases, the wearable device may detect that a user is performing an activity (e.g., an activity that meets a movement threshold, an activity resulting in a heart rate that meets a heart rate threshold), exercising, etc. and may perform the heart rate determination procedure 300 described herein based on detecting the activity. In some cases, a user may inform the wearable device that the user is performing such an activity or otherwise prompt the wearable device to perform the heart rate determination procedure 300 described herein.

At 305, the wearable device may measure motion data (e.g., acceleration data) associated with the user. In some cases, the wearable device may measure motion data constantly, periodically (e.g., in accordance with a periodicity), based on activity, time of day, etc. For example, the wearable device may be configured to measure motion data (e.g., a motion data point) every one second. In some cases, the wearable device may be configured to obtain the motion data and perform the procedure described herein based on the user of the wearable device performing an activity (e.g., such as an activity that meets a movement threshold), exercising, etc. During such an activity, the wearable device may be configured to measure the motion data in accordance with a periodicity, such as every one second for the duration of the activity.

In some cases, the wearable device may measure motion for a configured duration, where the wearable device may be configured with or receive an indication of the configured duration or may determine the configured duration. In some cases, the duration may be based on a duration of an activity, exercise, etc. being performed by the user. To measure motion data, the wearable device may utilize one or more motion sensors on the wearable device (e.g., motion sensors 245). In some cases, the motion data may refer to acceleration data. In such cases, the motion sensors on the wearable device may refer to accelerometers (e.g., 3D accelerometers) that are capable of detecting a user's acceleration, such as 3D acceleration. The wearable device may obtain motion data in the x, y, and z axis, where each axis may be referred to as a different motion channel. Accordingly, the wearable device may obtain motion data over three motion channels. In some cases, the accelerometers may measure the user's acceleration at 50 Hz, or some other frequency.

At 310, the wearable device, the user device, the servers, or any combination thereof, may preprocess the motion data. For example, the wearable device may preprocess the 3D acceleration data (e.g., raw data) to obtain processed acceleration data. The wearable device may preprocess each motion channel separately. In some cases, the preprocessing may include removing outliers, erroneous data, noise, or a combination thereof from the motion data.

At 315, the wearable device, the user device, the servers, or any combination thereof, may perform a multipath merger procedure. As described, the wearable device may obtain motion data via three channels: x, y, and z. Accordingly, the wearable device may input the data associated with the three separate channels into a multipath merger module to obtain a single channel (e.g., a single set of motion data). In some cases, the wearable device may be configured to select one motion channel from the set of motion channels (e.g., select the x-axis motion channel, the y-axis motion channel, or the z-axis motion channel). The selection may be based on quality, such that the wearable device may select the channel associated with the highest quality, the least number of outliers, the least amount of noise, or some combination thereof.

In some cases, the wearable device, the user device, the servers, or any combination thereof, may combine two or more motion channels to generate aggregate or composite motion data using any mathematical operation, including an averaging operation, a weighted averaging operation, and the like. For example, the wearable device may average at least two of the channels. In some cases, the wearable device may average the two channels associated with the highest quality (e.g., associated with the least amount of outliers, noise, etc.). In some cases, the wearable device may be configured to average any of the channels if each of the channels meets a quality threshold. In some cases, the wearable device may be configured to average a particular number of channels. For example, the wearable device may be configured to average all three of the motion channels to obtain the average channel. Accordingly, the output of the multipath merger module is a single motion channel. At 320, the wearable device, the user device, the servers, or any combination thereof, may input the motion data (e.g., the preprocessed and merged/composite motion data) into a time domain processor module. The time domain processor module may calculate an intensity associated with the motion data (e.g., motion intensity), a change in motion intensity (e.g., intensity change rate), or both. The time domain processor module may output motion intensity data over time, or change in motion intensity over time. In some cases, calculating the motion intensity and/or intensity change rate may include calculating an absolute value of the motion data, and calculating the mean of the absolute value. In some cases, the wearable device may input time-domain motion data into the time domain processor module and the module may output frequency-domain motion data, or vice versa. In other words, components of the system 200 may convert the motion data into a time/frequency domain representation of the motion data.

At 325, the wearable device may obtain PPG data. The wearable device may sample the PPG at a set of conditions, such as frequency (e.g., 50 Hz). In some cases, the wearable device may measure PPG data constantly, periodically (e.g., in accordance with a periodicity), based on activity, time of day, etc. For example, the wearable device may be configured to measure PPG data (e.g., collect a PPG data point) every one second. In some cases, the wearable device may be configured to obtain the PPG data and perform the procedure described herein based on the user of the wearable device performing an activity (e.g., such as during a time that an activity meets a movement threshold), exercising, etc. During such an activity, the wearable device may be configured to measure the PPG data in accordance with a periodicity, such as every one second for the duration of the activity.

In some cases, the wearable device may measure motion for a configured duration, where the wearable device may be configured with or receive an indication of the configured duration or may determine the configured duration. In some cases, the duration may be based on a duration of an activity, exercise, etc. being performed by the user. To measure PPG data, the wearable device may sample PPG data for the user using one or more sets of PPG sensors, where each set of PPG sensors includes at least one light source (e.g., LED) and at least one photodetector. For example, a wearable device may include a first pair of PPG sensors including a first LED and a first photodetector, and a second pair of PPG sensors including a second LED and a second photodetector. In other words, the wearable device may include two separate “channels” for acquiring PPG data (e.g., two separate PPG signals from the two respective pairs of sensors). In some cases, the wearable device may sample PPG data using the first and second pairs of PPG sensors simultaneously. Additionally, or alternatively, the wearable device may sample PPG data using the first and second sets of PPG sensors sequentially. For instance, the wearable device may sequentially control an activation state of the first pair of PPG sensors and the second pair of PPG sensors (e.g., first pair is in an active state when the second pair is in an inactive state, and vice versa).

In some implementations, the wearable device may sample PPG using components across multiple sets of sensors. For example, the wearable device may obtain PPG data using the first LED and the second photodetector, or using the second LED and the first photodetector, or both. In some cases, the first LED and the second photodetector may be opposite each other on the wearable device. Accordingly, in a wearable device that includes two pairs of PPG sensors, the wearable device may obtain four PPG signals (e.g., four PPG channels). Sequentially activating separate sets/pairs of PPG sensors may improve the quality and accuracy of each respective PPG signal, reduce interference, and may lead to more accurate heart rate measurements.

At 330, the wearable device, the user device, the servers, or any combination thereof, may preprocess the PPG data. For example, the wearable device may filter the PPG data, such as to remove erroneous samples, outliers, noise, etc. The wearable device may preprocess each PPG channel separately.

At 335, the wearable device, the user device, the servers, or any combination thereof, may perform a multipath merger procedure. As described, the wearable device may monitor PPG signals from multiple sets of sensors. For example, the wearable device may obtain four PPG signals (e.g., PPG signals collected via four separate channels). Accordingly, the wearable device may input the data associated with the four separate channels into a multipath merger module to obtain a single channel (e.g., a single set of PPG data).

In some cases, the wearable device may be configured to select one PPG channel from the set of PPG channels. The selection may be based on quality, such that the wearable device may select the channel associated with the highest quality, least number of outliers, noise, etc. For example, in the context of a ring wearable device, each of the respective sensors used for PPG sampling (e.g., LEDs, photodetectors) may be positioned at a different radial position along an inner circumference of the ring. Accordingly, one of the PPG signals from one or more of the multiple sets of PPG sensors may be more reliable than the PPG signals obtained from other of the multiple sensors, such as due to the positions of the sensors around the wearable device and in relation to the user, a relative quality of skin contact with each respective sensor or LED, and the like. For example, a sensor located on the underside (e.g., palm side) of a user's finger may result in more accurate PPG data than a sensor located over the bone of the finger (e.g., on the backhand side of the finger). By way of another example, a relative positioning of a wearable device may result in less skin contact at one first sensor as compared to a second sensor, and may therefore result in lower quality PPG data as compared to the second sensor. As such, the output of the multipath merger module may be based on a quality of the signal produced by each pair of sensors.

In some cases, the wearable device may combine two or more PPG channels/signals to generate aggregate or composite PPG data using any mathematical operation, including an averaging operation, a weighted averaging operation, and the like. For example, the wearable device may average at least two of the PPG channels. In some cases, the wearable device may average the top two or three PPG channels associated with the highest quality (e.g., associated with the least amount of outliers, noise, etc.). In some cases, the wearable device may be configured to average any number of the channels if each of the channels meets a quality threshold. In some cases, the wearable device may be configured to average a particular number of channels. For example, the wearable device may be configured to average all four of the PPG channels to obtain the average PPG channel. Accordingly, the output of the multipath merger module is a single PPG signal/channel (e.g., composite PPG signal). In some aspects, components of the system 200 may convert the PPG data (e.g., composite PPG signal) into a time/frequency domain representation of the PPG data.

At 340, the wearable device, the user device, the servers, or any combination thereof, may perform heart rate detection. The wearable device may estimate heart rate of the user based on the obtained PPG signal. Each time that a user's heart beats, blood is pumped out to the arteries located in the hands and fingers. PPG sensors (e.g., of the PPG system 235 as described with reference to FIG. 2) in the wearable device are able to detect these changes in blood flow and volume using light reflection and absorption. Each pulse causes the arteries in the finger to alternate between dilating and constricting (e.g., swelling and contracting). By shining a light on the skin, such as via LEDs, changes in light reflected back from the wavering volume of red blood cells in the arteries is measured. From here, PPG can represent these blood flow changes through a visual waveform that represents the activity of the user's heart, and thus heart rate. In some cases, the wearable device may determine the heart rate of the user based on the PPG signal satisfying a quality threshold, or based on portions of the PPG signal satisfying a quality threshold. For example, the wearable device may determine one or more portions of the PPG signal that actively represent the heart rate of the user.

As described herein, some activities may produce a PPG signal that may be misinterpreted as a heart rate signal. For example, exercises such as running, jumping, etc. may cause motion artifacts. The optical nature of PPG sensors may render the PPG measurements susceptible to motion artifacts from variable and discontinuous contact between the device and skin. Motion artifacts are typically caused by the change of blood flow velocity induced by the motion of the exercise or the relative movement between PPG sensors and skin of the user. For example, some motions may result in a periodic pressure between the wearable device (e.g., the sensors of the wearable device) and the part of the user that the wearable device is positioned, and as such may apply periodic pressure to blood vessels. The periodic pressure may cause the blood vessels to periodically constrict and dilate. Accordingly, the wearable device may detect such constriction and dilation of the vessels as an artificial PPG signal (e.g., motion artifacts) because the constriction and dilation were due to the motion of the user, and not due to the heart rate of the user.

Accordingly, to perform heart rate detection the wearable device may compare the PPG data and the motion data. For example, the wearable device may detect multiple strong PPG signals in a given time interval. The multiple PPG signals may represent multiple “candidate” heart rates for the user within the given time interval, where one of the candidate heart rates represents the user's actual heart rate, and other candidate heart rates may be attributable to motion (e.g., artificial heart rates, or motion artifacts). Accordingly, the system 200 may be configured to identify which candidate PPG signal represents the user's actual heart rate, and may be configured to eliminate or otherwise disregard PPG signals that are attributable to motion.

In some aspects, the system 200 may use the motion data such as the merged motion data, the motion intensity data, intensity change rate data, or a combination thereof to determine which of the multiple strong PPG signals (e.g., candidate PPG signals) are due to motion, and which PPG signal represents the user's actual heart rate. For example, the system 200 may determine that the motion data collected during a given time interval exhibits a relative periodicity (e.g., frequency pattern) which is similar or the same as a periodicity/frequency pattern exhibited by one of the candidate heart rates. In this example, the system 200 may identify the candidate heart rate that exhibits a similar periodicity/frequency pattern as the motion data as a motion artifact, and may therefore disregard the identified candidate heart rate/motion artifact for the purposes of identifying the user's actual heart rate. In some cases, the wearable device may analyze the motion data and the PPG data in units such that the wearable device may determine at which data points other PPG signals overlap with motion signals. The wearable device may remove the PPG signals that are overlapping with motion signals, or are otherwise attributable to motion (e.g., remove motion artifacts).

However, blood vessels of healthy humans are soft and elastic, and they have harmonic frequencies when waves travel through them. Harmonic frequencies are an integer multiple of the fundamental frequency. In some cases, some users (e.g., athletes) may exercise based on pulse harmonic frequency such that the user may match their cadence to a harmonic frequency of their pulse. Accordingly, in some cases, the wearable device may be unable to remove all PPG signals that are overlapping with a motion signal because it could be that the PPG signal that is overlapping is an actual heart rate signal to which the user accurately matched their cadence. Accordingly, upon determining overlapping PPG and motion signals, the wearable device may additionally analyze the data to determine whether to remove the overlapping PPG signals.

In some aspects, the system 200 may identify one or more candidate heart rates (e.g., candidate heart rate measurements) as motion artifacts based on a comparison of data trends between the candidate heart rate measurements and the motion data. For example, if a periodicity of motion data increases, the system 200 may identify a candidate heart rate measurement that shows an increasing heart rate as a motion artifact due to the comparison in trends between the motion data and the candidate heart rate data. In some cases, the wearable device may determine whether the motion data is steady, increasing, decreasing, etc. (e.g., trends in the heart rate data) and may use such information to determine dynamic heart rate limits (e.g., ranges). The wearable device may use the heart rate limits (e.g., expected range of human heart rates) to further determine which of the multiple PPG signals may be removed and determine the true heart rate signal. In other words, the wearable device may compare the PPG signals to the heart rate limits or ranges and may eliminate the PPG signals that do not fall within the expected heart rate limits or ranges (e.g., candidate heart rates with a periodicity/frequency pattern that falls outside an expected range of human heart rates may be labeled or identified as motion artifacts).

Upon determining the true heart rate signal (e.g., identifying the candidate PPG signal that represents the user's actual heart rate), the wearable device may detect gaps in the heart rate data. For example, with respect to a ring device, as the ring rotates, the PPG sensors located around the ring may also rotate and in some cases may be unable to detect a signal (e.g., such as due to the sensor being located over bone). Accordingly, in some cases, the wearable device may identify heart rate data on either side of the gap to interpolate the heart rate data in the gap. For example, the wearable device may analyze heart rate data during a first time (e.g., just prior to the gap), and analyze heart rate data during a second time (e.g., just after the gap). Based on the analysis, such as the trend, intensity, or both of the heart rate data and/or motion data during the first and second times, the wearable device may interpolate the heart rate data in the gap. For example, if an intensity of the motion data increased during the gap, it may be expected that the user's heart rate would also increase due to the increased motion intensity. As such, the system 200 may perform the interpolation across the gap to represent an increased heart rate due to the corresponding increase in motion intensity.

In additional or alternative implementations, the system 200 may utilize an “estimator” or a machine learning model/algorithm (e.g., heuristic-based model, deep learning model, regression-based model) to perform the heart rate detection at 340. In particular, the motion data collected/processed at 305 through 320, and the PPG data collected and processed at 325 through 335, may be inputted into a machine learning model, where the machine learning model is configured to determine a heart rate for the user (e.g., select or estimate a candidate heart rate from the set of candidate heart rates).

In some cases, in order to train a machine learning model for heart rate detection, wearable devices may acquire physiological data (e.g., PPG data, motion data) from one or more users (such as while the users are exercising), and ECG-based devices may be used to acquire heart rate data for the respective users (e.g., during the same time intervals). In such cases, the ECG-based heart rate data may serve as “ground truth” heart rate measurements that are used to train the machine learning model.

In this example, the physiological data (e.g., PPG data, heart rate data) may be inputted into the machine learning model, where the machine learning model is trained to generate or output the ECG-based heart rate data based on the received physiological data acquired from the wearable device(s). In other words, the ECG-based data may be considered a desired output of the model, where the machine learning model is trained to match (or closely estimate) the ECG-based heart rate data based on receiving the physiological data. As such, the machine learning model may be trained to receive PPG data and motion data as inputs, and generate heart rate measurements/estimations as outputs.

In some cases, the system 200 may train multiple versions of a machine learning model, such as for different demographics of users (e.g., different age groups, varying levels of activity/performance, different skin tones, etc.), for users with varying medical conditions, and the like. In this regard, different models which are tailored to different demographics of users may be used to further fine-tune the ability of the respective models to perform heart rate detection. For example, the system 200 may acquire physiological data from a user who is an avid runner, and may utilize a machine learning model trained on data from other runners to perform heart rate detection for the user.

After training the machine learning model(s), the machine learning model may be used to perform “live” heart rate measurements for users based on physiological data (e.g., PPG data, motion data) acquired from the wearable device. For example, time/frequency domain motion data and time/frequency PPG data illustrated in FIG. 3 may be inputted into the machine learning model at 340, where the machine learning model is configured to output a heart rate measurement/estimation based on the received data.

For instance, the machine learning model may be configured to differentiate between candidate heart rate measurements that are attributable to motion artifacts from candidate heart rate measurements that are indicative of the user's actual heart rate. In such cases, the machine learning model may be configured to select (e.g., identify, estimate) a candidate heart rate measurement, and determine a heart rate for the user based on the selected/estimated candidate heart rate measurements. For instance, the machine learning model may be configured to identify PPG heart rate candidates (e.g., candidate heart rate measurements) within the received heart rate data, and estimate a heart rate measurement based on the identified candidate heart rate (e.g., by selecting one of the candidate heart rate measurements, modifying a candidate heart rate measurement, estimating a new candidate heart rate measurement, etc.).

By way of another example, the machine learning model may be configured to identify or extract time-domain and/or frequency-domain features within the received PPG data and motion data. In this example, the time-domain and/or frequency-domain features may be used as inputs to the machine learning model to determine/estimate heart rate measurements. For instance, time/frequency domain features extracted from acquired physiological data may include the candidate heart rate measurements, where the machine learning model is configured to receive the candidate heart rate measurements as an input and estimate a heart rate measurement (e.g., select or modify a candidate heart rate measurement) as an output.

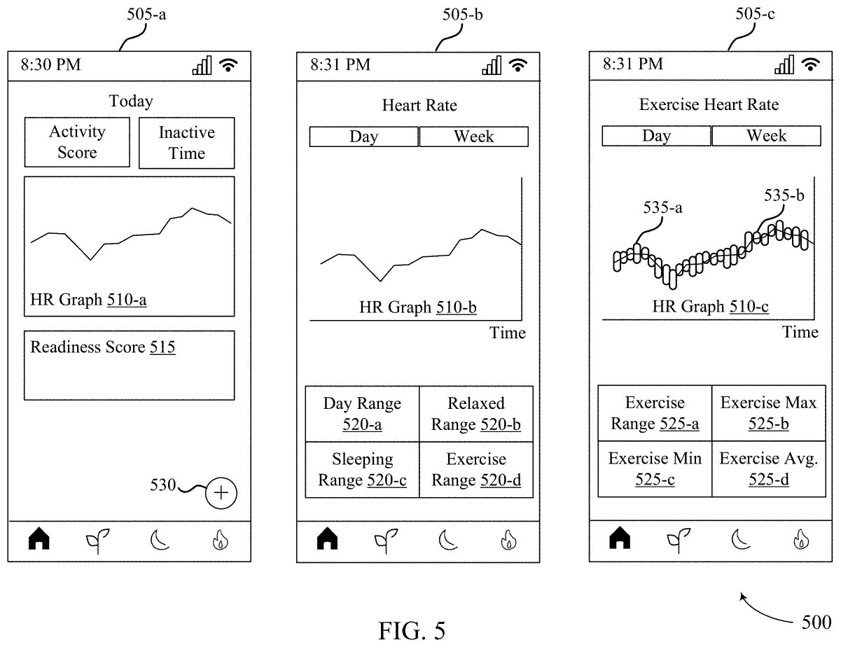

At 345, the wearable device may output the true heart rate data. The time series of the heart rate data may be displayed in an application (e.g., a GUI 275). Presentation of heart rate data will be further shown and described with reference to FIG. 5, below.

In some cases, steps 305 through 345 may be performed by the wearable device, a user device (e.g., user device 106), an application (e.g., an application of user device 106), or a combination thereof. For example, a combination of the wearable device and a user device may perform steps 305 through 345, and an application may output the heart rate data on the user device. In some cases, steps 305 through 335 may be performed every n seconds (e.g., such as every one second). Additionally, or alternatively, one or more aspects of step 340 may be performed in accordance with a configured periodicity. For example, the wearable device may detect strong motion signals every x seconds (e.g., 5 seconds), and detect strong PPG signals evert y seconds (e.g., 15 seconds). The wearable device may be configured to perform at least steps 305 through 335 during the activity, exercise, etc. and may be configured to perform one or more aspects of steps 340 and 345 post-activity, exercise, etc. In some cases, the wearable device may be configured to perform one or more aspects of steps 340 and 345 during the activity, exercise, etc.

FIG. 4 illustrates an example of a heart rate determination procedure 400 that supports techniques for measuring heart rate in accordance with aspects of the present disclosure. The heart rate determination procedure 400 may implement, or be implemented by, aspects of the system 100, system 200, or a combination thereof. In some cases, one or more aspects of heart rate determination procedure 400 may be the same or similar to one or more aspects of heart rate determination procedure 300. In some implementations, the heart rate determination procedure 400 may result in heart rate data (e.g., active heart rate data, exercise heart rate data) that may be displayed to a user via the GUI 275 of the user device 106, as shown in FIG. 2.

As described herein, a system, such a system 200, or a portion of system 200, such as a wearable device (e.g., a ring 104), may identify heart rate data of a user from a set of heart rate data, where the heart rate data may include motion artifacts. In some cases, a wearable device may detect heart rate data of a user in accordance with the techniques described herein during periods of activity, motion, and exercise. In some cases, the techniques for determining heart rate data described herein may be utilized for heart rate detections other than active heart rate detections, or for all heart rate detections (e.g., despite whether the user is active or exercising). Accordingly, the heart rate determination procedure may not be limited to determining heart rate of a user during exercise, motion, activity, etc., and may additionally or alternatively be used for determining heart rate during periods of rest, relaxation, mediation, and the like.

As described with reference to FIG. 3, the wearable device may obtain PPG data. To measure PPG data, the wearable device may sample PPG data for the user using one or more sets of PPG sensors, where each set of PPG sensor includes at least one LED and at least one photodetector. For example, in a wearable device that includes two pairs of PPG sensors, the wearable device may obtain four PPG signals (e.g., four PPG channels). For example, the wearable device may obtain PPG graphs 405-a, 405-b, 405-c, 405-d, which represent the PPG data obtained via the four different channels (e.g., the four different PPG sensor combinations).

The wearable device and/or other components of the system 200 may preprocess each PPG channel separately and subsequently may perform a multipath merger procedure so as to obtain a single PPG signal (e.g., composite PPG signal). In some cases, the wearable device may be configured to select one channel from the set of channels. The selection may be based on quality, such that the wearable device may select the channel associated with the highest quality, least number of outliers, noise, etc. In some cases, the wearable device may combine two or more PPG signals using one or more mathematical operations. For example, the wearable device may average at least two of the channels. In some cases, the wearable device may average the top two or three channels associated with the highest quality (e.g., associated with the least amount of outliers, noise, etc.). In some cases, the wearable device may be configured to average any number of the channels if each of the channels meets a quality threshold. In some cases, the wearable device may be configured to average a particular number of channels, or a particular subset of channels. The wearable device may be configured to average all four of the PPG channels to obtain the average PPG channel. In some cases, determining the single PPG channel may be based on one or more mathematical expressions. Accordingly, the output of the multipath merger module is a single PPG channel, such as PPG graph 405-e (e.g., a composite PPG signal).

Additionally, the wearable device may measure motion data (e.g., acceleration data) associated with the user. To measure motion data, the wearable device may utilize one or more motion sensors on the wearable device (e.g., motion sensors 245). In some cases, the wearable device may obtain motion data in the x, y, and z axis, where each axis may be referred to as a different motion channel. Accordingly, the wearable device may obtain motion data over three motion channels. With reference to FIG. 4, graphs 410-a, 410-b, and 410-c may represent motion data obtained in the x, y, and z axes, respectively. For example, graph 410-a may be associated with motion obtained in the x axis, graph 410-b may be associated with motion obtained in they axis, and graph 410-c may be associated with motion obtained in the z axis, for example. The wearable device may preprocess each of the channels, and then determine whether to combine one or more of the channels to obtain a single motion channel.

In some cases, the wearable device and/or other components of the system 200 may be configured to select one motion channel from the set of channels. The selection may be based on quality, such that the wearable device may select the channel associated with the highest quality, least number of outliers, noise, etc. In some cases, the wearable device may combine two or more of the motion channels using one or more mathematical operations. For example, the wearable device may average at least two of the channels. In some cases, the wearable device may average the two channels associated with the highest quality (e.g., associated with the least amount of outliers, noise, etc.). In some cases, the wearable device may be configured to average any of the channels if each of the channels meets a quality threshold. In some cases, the wearable device may be configured to average a particular number of channels, or a particular subset of channels. The wearable device may be configured to average all three of the motion channels to obtain the average channel. In some cases, determining the signal motion channel may be based on one or more mathematical expressions. Accordingly, upon selecting a single x, y, or z channel, or upon merging two of more of the channels, the wearable device may obtain a single motion graph 410-d (e.g., a composite motion signal).

Upon determining a single PPG signal and motion signal, the wearable device may determine a heart rate of the user. However, as noted previously herein, PPG data may be sensitive to motion, where in some cases, some activities or exercises may produce a PPG signal that is not due to heart rate. As such, the PPG signal may represent a false heart rate 425 (e.g., motion artifacts). For example, with reference to FIG. 4, graph 415 may depict an actual heart rate 420 and a false heart rate 425. Accordingly, to provide the user with accurate heart rate data, the wearable device may perform steps 320, 340, and 345 as described with reference to FIG. 3 to detect an actual heart rate signal of the user. In other words, the actual heart rate 420 and the false heart rate 425 may include “candidate” heart rate measurements, and the system 200 may be configured to determine whether the PPG data corresponding to the actual heart rate 420 or the PPG data corresponding to the false heart rate 425 accurately represents the user's real heart rate.

Accordingly, the wearable device may identify one or more heart rate measurements from the set of candidate heart rate measurements as motion artifacts based at least in part on a comparison of data trends between the set of candidate heart rate measurements (e.g., actual heart rate 420, false heart rate 425) and the motion data. The wearable device may select the first heart rate measurement from a subset of the set of candidate heart rate measurements that does not include the one or more heart rate measurements that were identified as motion artifacts. For example, the system 200 may identify the false heart rate 435 as a motion artifact, and may therefore select the actual heart rate 420 from the set of candidate heart rate measurements as the heart rate that reflects the user's actual heart rate during the given time interval.

Identifying the one or more heart rate measurements as motion artifacts may include identifying the one or more heart rate measurements from the set of candidate heart rate measurements as motion artifacts based at least in part on the one or more heart rate measurements exhibiting a similar frequency pattern relative to the motion data. As noted previously herein, the system 200 may use the motion data such as the merged motion data, the motion intensity data, intensity change rate data, or a combination thereof to determine which of the candidate heart rate measurements/PPG signals are due to motion, and which heart rate measurement/PPG signal represents the user's actual heart rate. In some cases, the wearable device may analyze the motion data and the PPG data in units such that the wearable device may determine at which data points other PPG signals overlap with motion signals. The wearable device may remove the PPG signals that are overlapping with motion signals, or are otherwise attributable to motion (e.g., remove motion artifacts).

As noted previously herein, in some implementations, the system 200 may utilize a machine learning model/algorithm (e.g., heuristic-based model, deep learning model, regression-based model) to perform the heart rate detection. In particular, a machine learning model may be trained to receive PPG data and motion data from a wearable device, and output a heart rate measurement/estimation based on the received data. For example, time/frequency domain PPG data (e.g., data including PPG heart rate candidates/candidate heart rates) and time/frequency domain motion data may be inputted into a machine learning model, where the machine learning model is configured to estimate/determine a heart rate measurement based on the received time/frequency domain PPG and motion data.

FIG. 5 illustrates an example of a GUI 500 that supports techniques for measuring heart rate in accordance with aspects of the present disclosure. The GUI 500 may implement, or be implemented by, aspects of the system 100, system 200, heart rate determination procedure 300, heart rate determination procedure 400, or any combination thereof. For example, the GUI 500 may include an example of the GUI 275 included within the user device 106 illustrated in FIG. 2.

The GUI 500 illustrates a series of application pages 505 that may be displayed to the user via the GUI 500 (e.g., GUI 275 illustrated in FIG. 2). The server 110 of system 200 may cause the GUI 500 of the user device 106 (e.g., mobile device) to display an indication of the heart rate data (e.g., via application page 505-a, or 505-b). Accordingly, upon determining heart rate data (e.g., as described with reference to FIGS. 3 and 4), the user may be presented with the application page 505-a upon opening the wearable application 250. As shown in FIG. 5, the application page 505-a may display a heart rate graph 510-a. The heart rate graph 510-a may include a visual representation of how the user's heart rate reacted to different events and activities (e.g., exercise, sleep, rest, etc.).

In some cases, the heart rate graph 510-a may display the heart rate of the user over minutes, hours, days, etc. In some cases, the heart rate graph 510 may display a combination of daytime heart rate data and nighttime heart rate data (e.g., awake heart rate data and asleep heart rate data). Additionally, in some implementations, the application page 505-a may display one or more scores (e.g., Sleep Score, Readiness Score 515, Activity Score, inactive time) for the user for the respective day (e.g., respective sleep day), where the one or more scores may be based on the heart rate data. By way of another example, the heart rate data may be used to update at least a subset of the factors for the Readiness Score 515 (e.g., subset of sleep, sleep balance, HRV balance, recovery index, activity, activity balance). In some cases, application page 505-a may include an addition button 530 that a user may press to add additional information to the page, such as a workout, a moment, a tag, etc.

Continuing with reference to FIG. 5, a user may be able to select the heart rate graph 510-a on the application page 505-a in order to view details associated with the heart rate, as shown in application page 505-b (“heart rate modal”). In other words, tapping on the heart rate graph 510-a shown on application page 505-a may cause the GUI 500 to display application page 505-b so that the user may quickly and easily view the heart rate of the user over time. The application page 505-b may include a modal view including details for the heart rate. Heart rate graphs 510-a and 510-b may display the same or different graph. For example, the time scale may be the same or different. In some cases, application page 505-b may indicate a portion of the heart rate graph 510-b associated with increased motion, exercise, activity, etc., such as to explain a reason for a change in heart rate. Application page 505-b may also include a day heart rate range 520-a, a relaxed heart rate range 520-b, a sleeping heart rate range 520-c, an exercise heart rate range 520-d, etc. The individual ranges may be on a per-hour basis, for example. In some cases, the application page 505-b may display the heart rate data as HRV, resting heart rate, etc.

In some cases, a user may be able to select the exercise range 520-d, or some other aspect of application page 505-b associated with exercise, motion, activity, etc. in order to view details associated with the heart rate during such activity, as shown in application page 505-c (“Exercise heart rate modal”). In other words, tapping on an aspect of the application page 505-b may cause the GUI 500 to display application page 505-c so that the user may quickly and easily view the heart rate of the user over time during a specific activity, such as the last exercise the user performed. The application page 505-c may include a modal view including details for the heart rate. In some cases, application page 505-c may display an exercise heart rate graph 510-c to represent how a user's rate changed during an exercise, activity, etc. In some cases, a duration of the heart rate graph 510-c may be based on an input from the user. For example, the user may add a workout (e.g., using button 530), where the user may indicate or the wearable device and/or application may otherwise determine a length of the workout, where the heart rate graph 510-c may be based on the length. Application page 505-c may also include an exercise heart rate range 525-a, a maximum exercise heart rate range 525-b, a minimum exercise heart rate 525-c, an average exercise heart rate 252-d, etc. The individual ranges may be on a per-workout basis, for example.

In some implementations, the system 200 may be configured to determine one or more accuracy metrics for determined heart rate measurements (e.g., “signal check” metrics). Accuracy metrics may include any metrics or predictors of how accurate and/or reliable a determined heart rate measurement is for a user. In some aspects, accuracy metrics may be determined or derived from a strength/power of candidate heart rate measurements, as well as a quantity of strong candidate heart rate measurements within some measurement period or time interval. In cases of low signal quality (e.g., due to the ring being too loose, motion artifacts too intense, etc.), the system 200 may not be able to confidently measure a user's heart rate, which may result in relatively low accuracy metrics (e.g., relatively low accuracy/reliability of heart rate measurements). In some implementations, the system 200 may be configured to discard, ignore, or otherwise refrain from displaying heart rate measurements with accuracy metrics that are less than some threshold metric (to avoid presenting erroneous heart rate values to the user). In other words, the system 200 may be configured to display only heart rate measurements that exhibit some threshold level of accuracy/reliability (e.g., heart rate measurements with accuracy metrics that satisfy some threshold metric).

The server of system may cause the GUI 500 of the user device to display a message on application pages 505-a, 505-b, 505-c, or a combination thereof, associated with the identified heart rate data. The user device may display recommendations and/or information associated with the heart rate data via a message. In some implementations, the user device 106 and/or servers 110 may generate alerts (e.g., messages, insights) associated with the heart rate data, which may be displayed to the user via the GUI 500 (e.g., application pages 505-a, 505-b, 50-c, or some other application page). In particular, the messages generated and displayed to the user via the GUI 500 may be associated with one or more characteristics (e.g., time of day, duration, range) of the heart rate data. For example, the message may alert the user to breathe, take a moment to relax, etc., based on the user's heart rate. In some cases, the message may alert the user to perform an exercise or activity because it has been a duration greater than a threshold since the user last performed an activity or exercise. In some cases, the message may display a recommendation to the user of how to adjust their lifestyle to achieve a particular heart rate. For example, the message may alert the user to target a particular heart rate during exercise, where the recommendation may be based on a type of workout the user is performing. In some cases, the message may alert the user to reduce exercise intensity to lower the user's heart rate, or to increase intensity to reach a target heart rate, etc. In this regard, the system may be configured to display messages or insights to the user in order to facilitate effective, healthy patterns for the user.

The heart rate graphs 510 may be illustrated via any visual representation, including line graphs, bar graphs, or any combination thereof. For example, the heart rate graphs 510-a and 510-b may include line graphs, where the heart rate graph 510-c may include a line graph overlaid with (or on top of) heart rate measurements 535 (e.g., heart rate measurements). In some implementations, the heart rate measurements 535, such as heart rate measurements 535-a and 535-b, may illustrate a range of potential heart rate measurements for a given period of time. In this regard, the relative height of the heart rate measurements may be associated with (or indicative of) a relative confidence metric for the respective heart rate measurement 535. For example, as shown in FIG. 5, the first heart rate measurement 535-a exhibits a larger height as compared to the second heart rate measurement 535-b, which may indicate that the system 200 was able to calculate the second heart rate measurement 535-b with a higher confidence metric (e.g., more accurate calculation) as compared to the first heart rate measurement 535-a.

In some implementations, the user device 106 may be configured to illustrate or otherwise indicate heart rate measurements 535 associated with the heart rate graphs 510 via haptic feedback, audio feedback, and the like. In particular, the user device 106 may be configured to provide haptic and/or audio feedback which corresponds to different heart rate measurements (e.g., heart rate measurements 535) as the user interacts with (e.g., touches, presses) different portions of the heart rate graphs 510.

For example, a user may slide their finger (or a mouse, stylus, etc.) along the heart rate graph 510-c to select different heart rate measurements 535 within the heart rate graph 510-c. In this example, the user device 106 may provide haptic feedback (or audio feedback) indicative of the selected heart rate measurements 535. For instance, in cases where the first heart rate measurement 535-a is 70 beats per minute, the user device 106 may provide haptic and/or audio feedback at a rate of 70 vibrations, beeps, chimes, etc., per minute when the user selects (e.g., presses and holds, clicks on, etc.) the first heart rate measurement 535-a (or a point on the heart rate graph 510-c corresponding to a point along the x-axis corresponding that is associated with the first heart rate measurement 535-a). Similarly, in cases where the second heart rate measurement 535-b is 85 beats per minute, the user device 106 may provide haptic and/or audio feedback at a rate of 85 vibrations, beeps, chimes, etc., per minute when the user selects (e.g., presses and holds, clicks on, etc.) the second heart rate measurement 535-b (or a point on the heart rate graph 510-c corresponding to a point along x-axis corresponding to the second heart rate measurement 535-b).

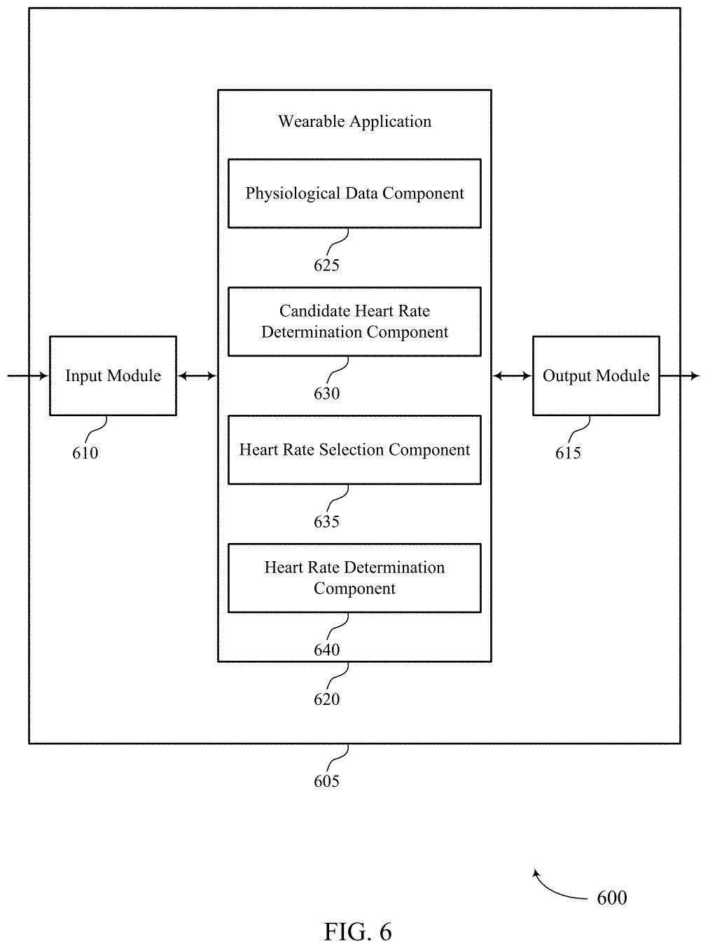

FIG. 6 shows a block diagram 600 of a device 605 that supports techniques for measuring heart rate in accordance with aspects of the present disclosure. The device 605 may include an input module 610, an output module 615, and a wearable application 620. The device 605 may also include a processor. Each of these components may be in communication with one another (e.g., via one or more buses).

The input module 610 may provide a means for receiving information such as packets, user data, control information, or any combination thereof associated with various information channels (e.g., control channels, data channels, information channels related to illness detection techniques). Information may be passed on to other components of the device 605. The input module 610 may utilize a single antenna or a set of multiple antennas.

The output module 615 may provide a means for transmitting signals generated by other components of the device 605. For example, the output module 615 may transmit information such as packets, user data, control information, or any combination thereof associated with various information channels (e.g., control channels, data channels, information channels related to illness detection techniques). In some examples, the output module 615 may be co-located with the input module 610 in a transceiver module. The output module 615 may utilize a single antenna or a set of multiple antennas.

For example, the wearable application 620 may include a physiological data component 625, a candidate heart rate determination component 630, a heart rate selection component 635, a heart rate determination component 640, or any combination thereof. In some examples, the wearable application 620, or various components thereof, may be configured to perform various operations (e.g., receiving, monitoring, transmitting) using or otherwise in cooperation with the input module 610, the output module 615, or both. For example, the wearable application 620 may receive information from the input module 610, send information to the output module 615, or be integrated in combination with the input module 610, the output module 615, or both to receive information, transmit information, or perform various other operations as described herein.

The physiological data component 625 may be configured as or otherwise support a means for receiving physiological data associated with the user, the physiological data comprising PPG data and motion data collected throughout a first time interval via a wearable device associated with the user. The candidate heart rate determination component 630 may be configured as or otherwise support a means for determining a set of candidate heart rate measurements within the first time interval based at least in part on the PPG data. The heart rate selection component 635 may be configured as or otherwise support a means for selecting a first heart rate measurement from the set of candidate heart rate measurements based at least in part on the received motion data. The heart rate determination component 640 may be configured as or otherwise support a means for determining a first heart rate for the user within the first time interval based at least in part on the selected first heart rate measurement.

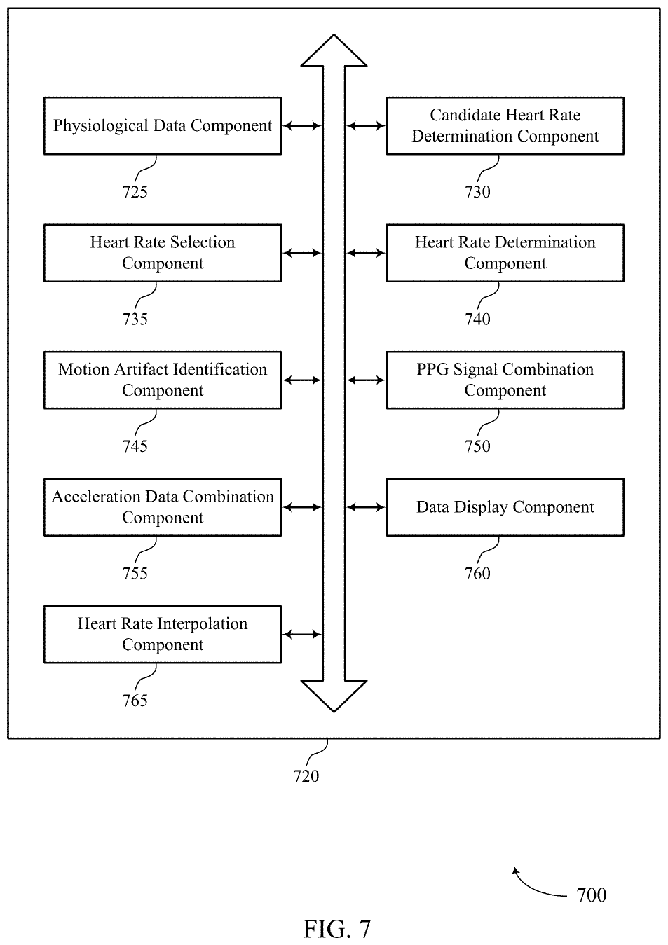

FIG. 7 shows a block diagram 700 of a wearable application 720 that supports techniques for measuring heart rate in accordance with aspects of the present disclosure. The wearable application 720 may be an example of aspects of a wearable application or a wearable application 620, or both, as described herein. The wearable application 720, or various components thereof, may be an example of means for performing various aspects of techniques for measuring heart rate as described herein. For example, the wearable application 720 may include a physiological data component 725, a candidate heart rate determination component 730, a heart rate selection component 735, a heart rate determination component 740, a motion artifact identification component 745, a PPG signal combination component 750, an acceleration data combination component 755, a data display component 760, a heart rate interpolation component 765, or any combination thereof. Each of these components may communicate, directly or indirectly, with one another (e.g., via one or more buses).

The physiological data component 725 may be configured as or otherwise support a means for receiving physiological data associated with the user, the physiological data comprising PPG data and motion data collected throughout a first time interval via a wearable device associated with the user. The candidate heart rate determination component 730 may be configured as or otherwise support a means for determining a set of candidate heart rate measurements within the first time interval based at least in part on the PPG data. The heart rate selection component 735 may be configured as or otherwise support a means for selecting a first heart rate measurement from the set of candidate heart rate measurements based at least in part on the received motion data. The heart rate determination component 740 may be configured as or otherwise support a means for determining a first heart rate for the user within the first time interval based at least in part on the selected first heart rate measurement.

In some examples, the motion artifact identification component 745 may be configured as or otherwise support a means for identifying one or more heart rate measurements from the set of candidate heart rate measurements as motion artifacts based at least in part on a comparison of data trends between the set of candidate heart rate measurements and the motion data. In some examples, the heart rate selection component 735 may be configured as or otherwise support a means for selecting the first heart rate measurement from a subset of the set of candidate heart rate measurements that does not include the one or more heart rate measurements which were identified as motion artifacts.

In some examples, to support identifying the one or more heart rate measurements as motion artifacts, the motion artifact identification component 745 may be configured as or otherwise support a means for identifying the one or more heart rate measurements from the set of candidate heart rate measurements as motion artifacts based at least in part on the one or more heart rate measurements exhibiting a similar frequency pattern relative to the motion data.

In some examples, the physiological data component 725 may be configured as or otherwise support a means for receiving additional physiological data associated with the user, the additional physiological data comprising additional PPG data and additional motion data collected throughout a second time interval via the wearable device, the second time interval subsequent to the first time interval. In some examples, the candidate heart rate determination component 730 may be configured as or otherwise support a means for determining an additional set of candidate heart rate measurements within the second time interval based at least in part on the additional PPG data. In some examples, the heart rate selection component 735 may be configured as or otherwise support a means for selecting a second heart rate measurement from the additional set of candidate heart rate measurements based at least in part on the received additional motion data. In some examples, the heart rate determination component 740 may be configured as or otherwise support a means for determining a second heart rate for the user within the second time interval based at least in part on the selected second heart rate measurement.

In some examples, the heart rate interpolation component 765 may be configured as or otherwise support a means for interpolating between the first heart rate measurement for the first time interval and the second heart rate measurement for the second time interval, wherein determining the first heart rate for the user within the first time interval, determining the second heart rate for the user within the second time interval, or both, is based at least in part on the interpolating.

In some examples, to support interpolating, the heart rate interpolation component 765 may be configured as or otherwise support a means for interpolating between the first heart rate measurement for the first time interval and the second heart rate measurement for the second time interval based at least in part on an intensity of the motion data collected throughout the first time interval, an intensity of the additional motion data collected throughout the second time interval, or both.

In some examples, the PPG data comprises a plurality of PPG signals acquired from a plurality of pairs of PPG sensors, and the PPG signal combination component 750 may be configured as or otherwise support a means for combining the plurality of PPG signals to generate a composite PPG signal using one or more mathematical operations, the one or more mathematical operations comprising an averaging operation, a weighted averaging operation, or both, wherein determining the set of heart rate data series is based at least in part on the composite PPG signal.

In some examples, the motion data comprises first acceleration data relative to a first direction, and the acceleration data combination component 755 may be configured as or otherwise support a means for combining the first acceleration data, the second acceleration data, the third acceleration data, or any combination thereof, using one or more mathematical operations based at least in part on one or more characteristics of the first acceleration data, the second acceleration data, or the third acceleration data, wherein the selection of the first heart rate data series from the set of heart rate data series is based at least in part on combining the first acceleration data, the second acceleration data, the third acceleration data, or any combination thereof.

In some examples, to support determining the set of candidate heart rate measurements, the candidate heart rate determination component 730 may be configured as or otherwise support a means for determining the set of candidate heart rate measurements within a frequency range that corresponds to an expected range of human heart rates.

In some examples, the data display component 760 may be configured as or otherwise support a means for causing a GUI of a user device associated with the user to display an indication of the first heart rate.

In some examples, the wearable device comprises a wearable ring device.

In some examples, the wearable device collects the physiological data from the user based on arterial blood flow.

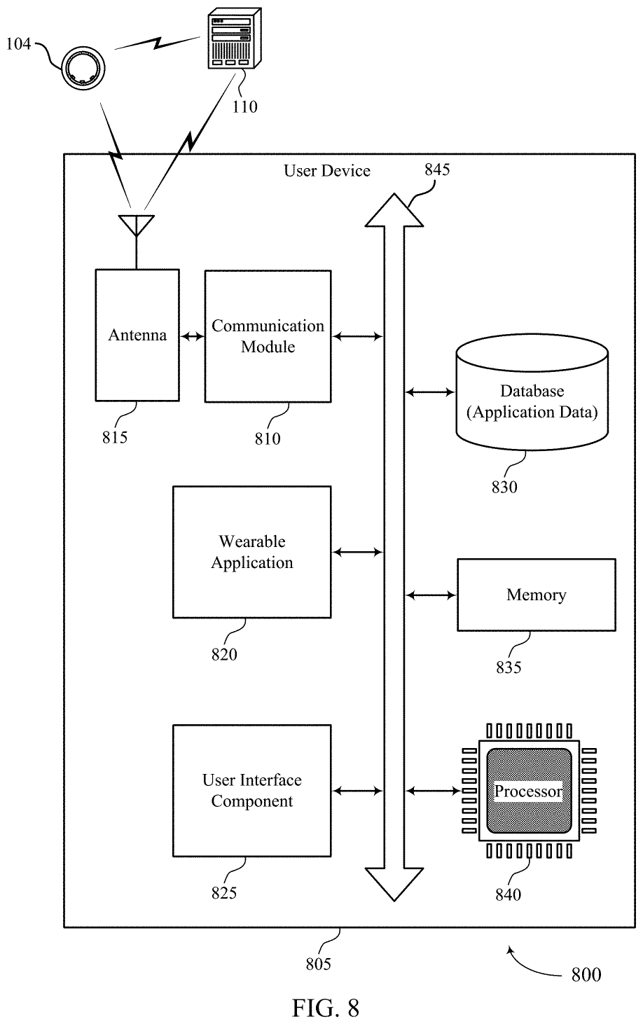

FIG. 8 shows a diagram of a system 800 including a device 805 that supports techniques for measuring heart rate in accordance with aspects of the present disclosure. The device 805 may be an example of or include the components of a device 605 as described herein. The device 805 may include an example of a user device 106, as described previously herein. The device 805 may include components for bi-directional communications including components for transmitting and receiving communications with a wearable device 104 and a server 110, such as a wearable application 820, a communication module 810, an antenna 815, a user interface component 825, a database (application data) 830, a memory 835, and a processor 840. These components may be in electronic communication or otherwise coupled (e.g., operatively, communicatively, functionally, electronically, electrically) via one or more buses (e.g., a bus 845).

The communication module 810 may manage input and output signals for the device 805 via the antenna 815. The communication module 810 may include an example of the communication module 220-b of the user device 106 shown and described in FIG. 2. In this regard, the communication module 810 may manage communications with the ring 104 and the server 110, as illustrated in FIG. 2. The communication module 810 may also manage peripherals not integrated into the device 805. In some cases, the communication module 810 may represent a physical connection or port to an external peripheral. In some cases, the communication module 810 may utilize an operating system such as iOS®, ANDROID®, MS-DOS®, MS-WINDOWS®, OS/2®, UNIX®, LINUX®, or another known operating system. In other cases, the communication module 810 may represent or interact with a wearable device (e.g., ring 104), modem, a keyboard, a mouse, a touchscreen, or a similar device. In some cases, the communication module 810 may be implemented as part of the processor 840. In some examples, a user may interact with the device 805 via the communication module 810, user interface component 825, or via hardware components controlled by the communication module 810.

In some cases, the device 805 may include a single antenna 815. However, in some other cases, the device 805 may have more than one antenna 815, which may be capable of concurrently transmitting or receiving multiple wireless transmissions. The communication module 810 may communicate bi-directionally, via the one or more antennas 815, wired, or wireless links as described herein. For example, the communication module 810 may represent a wireless transceiver and may communicate bi-directionally with another wireless transceiver. The communication module 810 may also include a modem to modulate the packets, to provide the modulated packets to one or more antennas 815 for transmission, and to demodulate packets received from the one or more antennas 815.

The user interface component 825 may manage data storage and processing in a database 830. In some cases, a user may interact with the user interface component 825. In other cases, the user interface component 825 may operate automatically without user interaction. The database 830 may be an example of a single database, a distributed database, multiple distributed databases, a data store, a data lake, or an emergency backup database.

The memory 835 may include RAM and ROM. The memory 835 may store computer-readable, computer-executable software including instructions that, when executed, cause the processor 840 to perform various functions described herein. In some cases, the memory 835 may contain, among other things, a basic I/O system (BIOS) which may control basic hardware or software operation such as the interaction with peripheral components or devices.

The processor 840 may include an intelligent hardware device, (e.g., a general-purpose processor, a digital signal processor (DSP), a central processing unit (CPU), a microcontroller, an application-specific integrated circuit (ASIC), an field-programmable gate array (FPGA), a programmable logic device, a discrete gate or transistor logic component, a discrete hardware component, or any combination thereof). In some cases, the processor 840 may be configured to operate a memory array using a memory controller. In other cases, a memory controller may be integrated into the processor 840. The processor 840 may be configured to execute computer-readable instructions stored in a memory 835 to perform various functions (e.g., functions or tasks supporting a method and system for sleep staging algorithms).

For example, the wearable application 820 may be configured as or otherwise support a means for receiving physiological data associated with the user, the physiological data comprising PPG data and motion data collected throughout a first time interval via a wearable device associated with the user. The wearable application 820 may be configured as or otherwise support a means for determining a set of candidate heart rate measurements within the first time interval based at least in part on the PPG data. The wearable application 820 may be configured as or otherwise support a means for selecting a first heart rate measurement from the set of candidate heart rate measurements based at least in part on the received motion data. The wearable application 820 may be configured as or otherwise support a means for determining a first heart rate for the user within the first time interval based at least in part on the selected first heart rate measurement.

By including or configuring the wearable application 820 in accordance with examples as described herein, the device 805 may support techniques for improved heart rate data for a user, and improved alerts or instructions provided to a user.

The wearable application 820 may include an application (e.g., “app”), program, software, or other component that is configured to facilitate communications with a ring 104, server 110, other user devices 106, and the like. For example, the wearable application 820 may include an application executable on a user device 106 that is configured to receive data (e.g., physiological data) from a ring 104, perform processing operations on the received data, transmit and receive data with the servers 110, and cause presentation of data to a user 102.

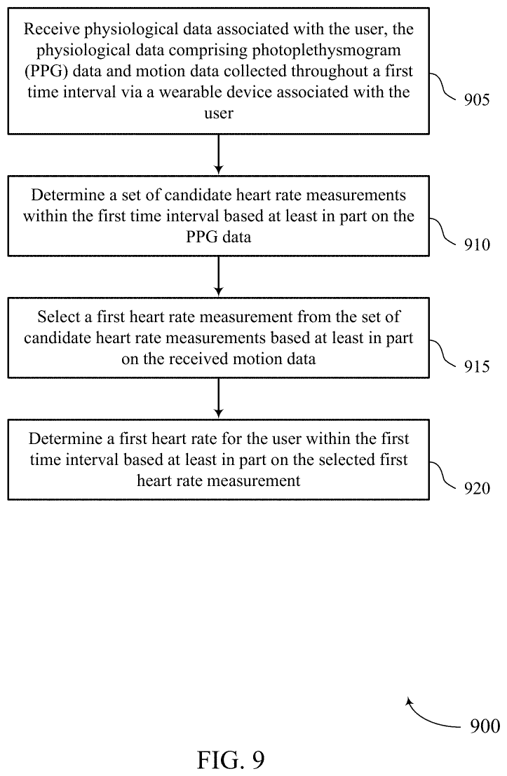

FIG. 9 shows a flowchart illustrating a method 900 that supports techniques for measuring heart rate in accordance with aspects of the present disclosure. The operations of the method 900 may be implemented by a user device or its components as described herein. For example, the operations of the method 900 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 905, the method may include receiving physiological data associated with the user, the physiological data comprising PPG data and motion data collected throughout a first time interval via a wearable device associated with the user. The operations of 905 may be performed in accordance with examples as disclosed herein. In some examples, aspects of the operations of 905 may be performed by a physiological data component 725 as described with reference to FIG. 7.

At 910, the method may include determining a set of candidate heart rate measurements within the first time interval based at least in part on the PPG data. The operations of 910 may be performed in accordance with examples as disclosed herein. In some examples, aspects of the operations of 910 may be performed by a candidate heart rate determination component 730 as described with reference to FIG. 7.

At 915, the method may include selecting a first heart rate measurement from the set of candidate heart rate measurements based at least in part on the received motion data. The operations of 915 may be performed in accordance with examples as disclosed herein. In some examples, aspects of the operations of 915 may be performed by a heart rate selection component 735 as described with reference to FIG. 7.

At 920, the method may include determining a first heart rate for the user within the first time interval based at least in part on the selected first heart rate measurement. The operations of 920 may be performed in accordance with examples as disclosed herein. In some examples, aspects of the operations of 920 may be performed by a heart rate determination component 740 as described with reference to FIG. 7.

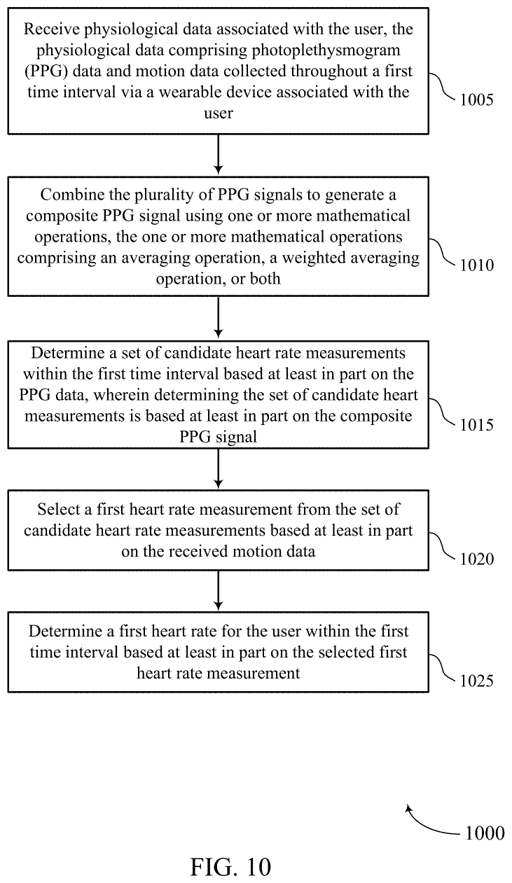

FIG. 10 shows a flowchart illustrating a method 1000 that supports techniques for measuring heart rate in accordance with aspects of the present disclosure. The operations of the method 1000 may be implemented by a user device or its components as described herein. For example, the operations of the method 1000 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 1005, the method may include receiving physiological data associated with the user, the physiological data comprising PPG data and motion data collected throughout a first time interval via a wearable device associated with the user. The operations of 1005 may be performed in accordance with examples as disclosed herein. In some examples, aspects of the operations of 1005 may be performed by a physiological data component 725 as described with reference to FIG. 7.

At 1010, the method may include combining the plurality of PPG signals to generate a composite PPG signal using one or more mathematical operations, the one or more mathematical operations comprising an averaging operation, a weighted averaging operation, or both. The operations of 1010 may be performed in accordance with examples as disclosed herein. In some examples, aspects of the operations of 1010 may be performed by a PPG signal combination component 750 as described with reference to FIG. 7.

At 1015, the method may include determining a set of candidate heart rate measurements within the first time interval based at least in part on the PPG data, wherein determining the set of candidate heart rate measurements is based at least in part on the composite PPG signal. The operations of 1015 may be performed in accordance with examples as disclosed herein. In some examples, aspects of the operations of 1015 may be performed by a candidate heart rate determination component 730 as described with reference to FIG. 7.

At 1020, the method may include selecting a first heart rate measurement from the set of candidate heart rate measurements based at least in part on the received motion data. The operations of 1020 may be performed in accordance with examples as disclosed herein. In some examples, aspects of the operations of 1020 may be performed by a heart rate selection component 735 as described with reference to FIG. 7.

At 1025, the method may include determining a first heart rate for the user within the first time interval based at least in part on the selected first heart rate measurement. The operations of 1025 may be performed in accordance with examples as disclosed herein. In some examples, aspects of the operations of 1025 may be performed by a heart rate determination component 740 as described with reference to FIG. 7.

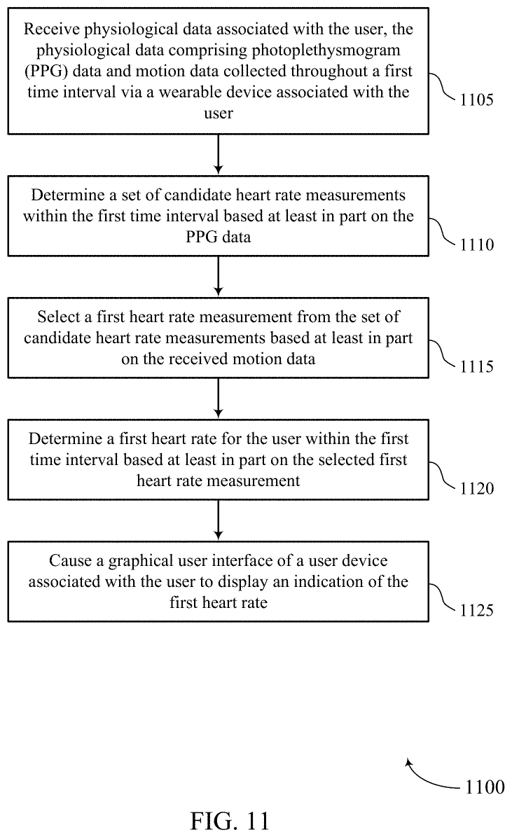

FIG. 11 shows a flowchart illustrating a method 1100 that supports techniques for measuring heart rate in accordance with aspects of the present disclosure. The operations of the method 1100 may be implemented by a user device or its components as described herein. For example, the operations of the method 1100 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 1105, the method may include receiving physiological data associated with the user, the physiological data comprising PPG data and motion data collected throughout a first time interval via a wearable device associated with the user. The operations of 1105 may be performed in accordance with examples as disclosed herein. In some examples, aspects of the operations of 1105 may be performed by a physiological data component 725 as described with reference to FIG. 7.

At 1110, the method may include determining a set of candidate heart rate measurements within the first time interval based at least in part on the PPG data. The operations of 1110 may be performed in accordance with examples as disclosed herein. In some examples, aspects of the operations of 1110 may be performed by a candidate heart rate determination component 730 as described with reference to FIG. 7.

At 1115, the method may include selecting a first heart rate measurement from the set of candidate heart rate measurements based at least in part on the received motion data. The operations of 1115 may be performed in accordance with examples as disclosed herein. In some examples, aspects of the operations of 1115 may be performed by a heart rate selection component 735 as described with reference to FIG. 7.

At 1120, the method may include determining a first heart rate for the user within the first time interval based at least in part on the selected first heart rate measurement. The operations of 1120 may be performed in accordance with examples as disclosed herein. In some examples, aspects of the operations of 1120 may be performed by a heart rate determination component 740 as described with reference to FIG. 7.

At 1125, the method may include causing a GUI of a user device associated with the user to display an indication of the first heart rate. The operations of 1125 may be performed in accordance with examples as disclosed herein. In some examples, aspects of the operations of 1125 may be performed by a data display component 760 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method is described. The method may include receiving physiological data associated with the user, the physiological data comprising PPG data and motion data collected throughout a first time interval via a wearable device associated with the user, determining a set of candidate heart rate measurements within the first time interval based at least in part on the PPG data, selecting a first heart rate measurement from the set of candidate heart rate measurements based at least in part on the received motion data, and determining a first heart rate for the user within the first time interval based at least in part on the selected first heart rate measurement.

An apparatus is described. The apparatus may include a processor, memory coupled with the processor, and instructions stored in the memory. The instructions may be executable by the processor to cause the apparatus to receive physiological data associated with the user, the physiological data comprising PPG data and motion data collected throughout a first time interval via a wearable device associated with the user, determine a set of candidate heart rate measurements within the first time interval based at least in part on the PPG data, select a first heart rate measurement from the set of candidate heart rate measurements based at least in part on the received motion data, and determine a first heart rate for the user within the first time interval based at least in part on the selected first heart rate measurement.

Another apparatus is described. The apparatus may include means for receiving physiological data associated with the user, the physiological data comprising PPG data and motion data collected throughout a first time interval via a wearable device associated with the user, means for determining a set of candidate heart rate measurements within the first time interval based at least in part on the PPG data, means for selecting a first heart rate measurement from the set of candidate heart rate measurements based at least in part on the received motion data, and means for determining a first heart rate for the user within the first time interval based at least in part on the selected first heart rate measurement.

A non-transitory computer-readable medium storing code is described. The code may include instructions executable by a processor to receive physiological data associated with the user, the physiological data comprising PPG data and motion data collected throughout a first time interval via a wearable device associated with the user, determine a set of candidate heart rate measurements within the first time interval based at least in part on the PPG data, select a first heart rate measurement from the set of candidate heart rate measurements based at least in part on the received motion data, and determine a first heart rate for the user within the first time interval based at least in part on the selected first heart rate measurement.

Some examples of the method, apparatuses, and non-transitory computer-readable medium described herein may further include operations, features, means, or instructions for identifying one or more heart rate measurements from the set of candidate heart rate measurements as motion artifacts based at least in part on a comparison of data trends between the set of candidate heart rate measurements and the motion data and selecting the first heart rate measurement from a subset of the set of candidate heart rate measurements that does not include the one or more heart rate measurements which were identified as motion artifacts.

In some examples of the method, apparatuses, and non-transitory computer-readable medium described herein, identifying the one or more heart rate measurements as motion artifacts may include operations, features, means, or instructions for identifying the one or more heart rate measurements from the set of candidate heart rate measurements as motion artifacts based at least in part on the one or more heart rate measurements exhibiting a similar frequency pattern relative to the motion data.

Some examples of the method, apparatuses, and non-transitory computer-readable medium described herein may further include operations, features, means, or instructions for receiving additional physiological data associated with the user, the additional physiological data comprising additional PPG data and additional motion data collected throughout a second time interval via the wearable device, the second time interval subsequent to the first time interval, determining an additional set of candidate heart rate measurements within the second time interval based at least in part on the additional PPG data, selecting a second heart rate measurement from the additional set of candidate heart rate measurements based at least in part on the received additional motion data, and determining a second heart rate for the user within the second time interval based at least in part on the selected second heart rate measurement.

Some examples of the method, apparatuses, and non-transitory computer-readable medium described herein may further include operations, features, means, or instructions for interpolating between the first heart rate measurement for the first time interval and the second heart rate measurement for the second time interval, wherein determining the first heart rate for the user within the first time interval, determining the second heart rate for the user within the second time interval, or both, may be based at least in part on the interpolating.

In some examples of the method, apparatuses, and non-transitory computer-readable medium described herein, the interpolating may include operations, features, means, or instructions for interpolating between the first heart rate measurement for the first time interval and the second heart rate measurement for the second time interval based at least in part on an intensity of the motion data collected throughout the first time interval, an intensity of the additional motion data collected throughout the second time interval, or both.

In some examples of the method, apparatuses, and non-transitory computer-readable medium described herein, the PPG data comprises a plurality of PPG signals acquired from a plurality of pairs of PPG sensors and the method, apparatuses, and non-transitory computer-readable medium may include further operations, features, means, or instructions for combining the plurality of PPG signals to generate a composite PPG signal using one or more mathematical operations, the one or more mathematical operations comprising an averaging operation, a weighted averaging operation, or both, wherein determining the set of heart rate data series may be based at least in part on the composite PPG signal.

In some examples of the method, apparatuses, and non-transitory computer-readable medium described herein, the motion data comprises first acceleration data relative to a first direction and the method, apparatuses, and non-transitory computer-readable medium may include further operations, features, means, or instructions for combining the first acceleration data, the second acceleration data, the third acceleration data, or any combination thereof, using one or more mathematical operations based at least in part on one or more characteristics of the first acceleration data, the second acceleration data, or the third acceleration data, wherein the selection of the first heart rate data series from the set of heart rate data series may be based at least in part on combining the first acceleration data, the second acceleration data, the third acceleration data, or any combination thereof.

In some examples of the method, apparatuses, and non-transitory computer-readable medium described herein, determining the set of candidate heart rate measurements may include operations, features, means, or instructions for determining the set of candidate heart rate measurements within a frequency range that corresponds to an expected range of human heart rates.

Some examples of the method, apparatuses, and non-transitory computer-readable medium described herein may further include operations, features, means, or instructions for causing a GUI of a user device associated with the user to display an indication of the first heart rate.

In some examples of the method, apparatuses, and non-transitory computer-readable medium described herein, the wearable device comprises a wearable ring device.

In some examples of the method, apparatuses, and non-transitory computer-readable medium described herein, the wearable device collects the physiological data from the user based on arterial blood flow.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for measuring heart rate for a user, comprising:
receiving physiological data associated with the user, the physiological data comprising photoplethysmogram (PPG) data and motion data collected throughout a first time interval via a wearable device associated with the user;
determining a set of candidate heart rate measurements within the first time interval based at least in part on the PPG data;
selecting a first heart rate measurement from the set of candidate heart rate measurements based at least in part on the received motion data; and
determining a first heart rate for the user within the first time interval based at least in part on the selected first heart rate measurement.

2. The method of claim 1, further comprising:
inputting the PPG data and the motion data into a machine learning model, wherein selecting the first heart rate measurement, determining the first heart rate, or both, is based at least in part on inputting the PPG data and the motion data into the machine learning model.

3. The method of claim 1, further comprising:
identifying one or more heart rate measurements from the set of candidate heart rate measurements as motion artifacts based at least in part on a comparison of data trends between the set of candidate heart rate measurements and the motion data; and
selecting the first heart rate measurement from a subset of the set of candidate heart rate measurements that does not include the one or more heart rate measurements that were identified as motion artifacts.

4. The method of claim 3, wherein identifying the one or more heart rate measurements as motion artifacts comprises:
identifying the one or more heart rate measurements from the set of candidate heart rate measurements as motion artifacts based at least in part on the one or more heart rate measurements exhibiting a similar frequency pattern relative to the motion data.

5. The method of claim 1, further comprising:
receiving additional physiological data associated with the user, the additional physiological data comprising additional PPG data and additional motion data collected throughout a second time interval via the wearable device, the second time interval subsequent to the first time interval;
determining an additional set of candidate heart rate measurements within the second time interval based at least in part on the additional PPG data;
selecting a second heart rate measurement from the additional set of candidate heart rate measurements based at least in part on the received additional motion data; and
determining a second heart rate for the user within the second time interval based at least in part on the selected second heart rate measurement.

6. The method of claim 5, further comprising:
interpolating between the first heart rate measurement for the first time interval and the second heart rate measurement for the second time interval, wherein determining the first heart rate for the user within the first time interval, determining the second heart rate for the user within the second time interval, or both, is based at least in part on the interpolating.

7. The method of claim 5, wherein the interpolating comprises:
interpolating between the first heart rate measurement for the first time interval and the second heart rate measurement for the second time interval based at least in part on an intensity of the motion data collected throughout the first time interval, an intensity of the additional motion data collected throughout the second time interval, or both.

8. The method of claim 1, wherein the PPG data comprises a plurality of PPG signals acquired from a plurality of pairs of PPG sensors, wherein each pair of PPG sensors comprises at least one light-emitting diode and at least one photodetector, the method further comprising:
combining the plurality of PPG signals to generate a composite PPG signal using one or more mathematical operations, the one or more mathematical operations comprising an averaging operation, a weighted averaging operation, or both, wherein determining the set of candidate heart rate measurements is based at least in part on the composite PPG signal.

9. The method of claim 1, wherein the motion data comprises first acceleration data relative to a first direction, second acceleration data relative to a second direction, and third acceleration data relative to a third direction, the method further comprising:
combining the first acceleration data, the second acceleration data, the third acceleration data, or any combination thereof, using one or more mathematical operations based at least in part on one or more characteristics of the first acceleration data, the second acceleration data, or the third acceleration data, wherein the selection of the first heart rate measurement from the set of candidate heart rate measurements is based at least in part on combining the first acceleration data, the second acceleration data, the third acceleration data, or any combination thereof.

10. The method of claim 1, wherein determining the set of candidate heart rate measurements comprises:
determining the set of candidate heart rate measurements within a frequency range that corresponds to an expected range of human heart rates.

11. The method of claim 1, further comprising:
causing a graphical user interface of a user device associated with the user to display an indication of the first heart rate.

12. The method of claim 1, wherein the wearable device comprises a wearable ring device.

13. The method of claim 1, wherein the wearable device collects the physiological data from the user based on arterial blood flow.

14. An apparatus for measuring heart rate for a user, comprising:
a processor;
memory coupled with the processor; and
instructions stored in the memory and executable by the processor to cause the apparatus to:
receive physiological data associated with the user, the physiological data comprising photoplethysmogram (PPG) data and motion data collected throughout a first time interval via a wearable device associated with the user;
determine a set of candidate heart rate measurements within the first time interval based at least in part on the PPG data;
select a first heart rate measurement from the set of candidate heart rate measurements based at least in part on the received motion data; and
determine a first heart rate for the user within the first time interval based at least in part on the selected first heart rate measurement.

15. The apparatus of claim 14, wherein the instructions are further executable by the processor to cause the apparatus to:
identify one or more heart rate measurements from the set of candidate heart rate measurements as motion artifacts based at least in part on a comparison of data trends between the set of candidate heart rate measurements and the motion data; and
select the first heart rate measurement from a subset of the set of candidate heart rate measurements that does not include the one or more heart rate measurements that were identified as motion artifacts.

16. The apparatus of claim 15, wherein the instructions to identify the one or more heart rate measurements as motion artifacts are executable by the processor to cause the apparatus to:
identify the one or more heart rate measurements from the set of candidate heart rate measurements as motion artifacts based at least in part on the one or more heart rate measurements exhibiting a similar frequency pattern relative to the motion data.

17. The apparatus of claim 14, wherein the instructions are further executable by the processor to cause the apparatus to:
receive additional physiological data associated with the user, the additional physiological data comprising additional PPG data and additional motion data collected throughout a second time interval via the wearable device, the second time interval subsequent to the first time interval;
determine an additional set of candidate heart rate measurements within the second time interval based at least in part on the additional PPG data;
select a second heart rate measurement from the additional set of candidate heart rate measurements based at least in part on the received additional motion data; and
determine a second heart rate for the user within the second time interval based at least in part on the selected second heart rate measurement.

18. The apparatus of claim 17, wherein the instructions are further executable by the processor to cause the apparatus to:
interpolate between the first heart rate measurement for the first time interval and the second heart rate measurement for the second time interval, wherein determining the first heart rate for the user within the first time interval, determining the second heart rate for the user within the second time interval, or both, is based at least in part on the interpolating.

19. The apparatus of claim 17, wherein the instructions to interpolate are executable by the processor to cause the apparatus to:
interpolate between the first heart rate measurement for the first time interval and the second heart rate measurement for the second time interval based at least in part on an intensity of the motion data collected throughout the first time interval, an intensity of the additional motion data collected throughout the second time interval, or both.

20. The apparatus of claim 14, wherein the PPG data comprises a plurality of PPG signals acquired from a plurality of pairs of PPG sensors, and the instructions are further executable by the processor to cause the apparatus to:
combine the plurality of PPG signals to generate a composite PPG signal using one or more mathematical operations, the one or more mathematical operations comprising an averaging operation, a weighted averaging operation, or both, wherein determining the set of candidate heart rate measurements is based at least in part on the composite PPG signal.
